(19) United States
(12) Patent Application Publication
(10) Pub. No.: US 2011/0045089 A1
(43) Pub. Date: Feb. 24, 2011

(54) VITAMIN K3 DERIVATIVE / NSA FORMULATION

(76) Inventors: Alexander Lieb, Brig (CH); Steffen Knapp, Schppheim (DE); Kamyab Amouzegar, Mount - Royal (CA)

Correspondence Address: Ronald J. Baron, Esq.;HOFFMANN & BARON, LLP, 6900 Jericho Turnpike, Syosset, NY, 11791, US

(21) Appl. No.: 12/850,220

(22) Filed: Aug. 4, 2010

Related U.S. Application Data
(60) Provisional application No. 61/231,380, filed on Aug. 5, 2009.

(30) Foreign Application Priority Data
Aug. 5, 2009 (EP) 09010104.9

(51) Int. Cl. A61K 9/00; A61K 31/4406; A61P 39/00; A61K 31/122

(52) U.S. Cl. 424/490; 514/356; 514/682

(57) ABSTRACT

The present invention discloses highly stabilized nicotinamide (NA) formulated vitamin K3 derivative particles, whereby the NA forms a physical protective layer (both continuous and discontinuous) leading to highly stabilized vitamin K3 derivative particles, as well as a process for their production.

### CROSS-REFERENCE TO RELATED APPLICATIONS AND CLAIM OF PRIORITY

This application claims the benefit of priority from European Patent Application No. 09010104.9 filed Aug. 5, 2009, and U.S. Provisional Patent Application No. 61/231,380 filed Aug. 5, 2009, the disclosures of which are incorporated herein by reference.

### BACKGROUND OF THE INVENTION

The present invention discloses highly stabilized nicotinamide (NA) formulated vitamin K3 derivative particles, whereby the NA forms a physical protective layer (both continuous and discontinuous) leading to highly stabilized vitamin K3 derivative particles, as well as a process for their production.

Vitamin K3 (2-methyl-1,4-naphthochinone; menadione) derivatives are used as an ingredient in the animal feed industry. Vitamin K3 derivatives were continuously developed in order to increase the stability of the vitamin. In the 1940s MSB (Menadione Sodium Bisulphite), which possesses a relatively low stability, was introduced in the market. A decade later, MSBC, a bisulphite complex consisting of MSB, NaHSO₃ and water, was offered as a new product form. The stability of this product was slightly higher, but not enough to guarantee a storage time of 4-6 months. In the 1960s it was shown that Menadione bisulfite adducts of substituted pyrimidines (such as 2-hydroxy-4,6-dimethyl-pyrimidinium Menadione Bisulfite or MPB) present higher stability in high humidity and high temperature compared to MSB (U.S. Pat. No. 3,328,169). During the 1970s, it was shown that when MSB is reacted with nicotinamide (instead of its reaction with 2-hydroxy-4,6-dimethyl-pyrimidine in the case of MPB), the obtained organic bisulfite adduct (Nicotinamide Menadione Bisulfite or MNB) is also a stabilized form of vitamin K3 with the advantage of replacing an inert component with a compound having a vitaminic activity (U.S. Pat. No. 4,577,019 and UK patent 2025976).

In all the examples provided in these patents, each mole of MSB is reacted with 1 to 3 moles of nicotinamide to precipitate 1 mole of MNB (which contains 1 mole of Menadione and 1 mole of nicotinamide). The sole reason for this use of excess amount of nicotinamide is to increase the precipitation yield. However, there is no indication in the prior art that excess amount of NA can be used to modify the composition of the final MNB product, which is made by a chemical reaction in which the sodium cation in the MSB molecule is replaced by a protonated nicotinamide molecule and the formation of MNB which precipitates due to its much lower water solubility.

The final MNB product is made by a stoechiometric chemical reaction in which the sodium cation in the MSB molecule is replaced by a protonated nicotinamide molecule. The use of excess amount of nicotinamide also increases the formation of MNB which precipitates due to its much lower solubility.

Nowadays MNB is the most stable product in the market and exclusively used for example in broiler pellets. However, even during this pelleting process (80° C., high humidity), 50% of MNB is decomposed. Even in the case of premixes containing choline chloride, although MNB is considered the most stable product, the premix does not contain more than 70% of the original amount of K3 after 6 months of storage as shown in FIG. 2 (data from M. Coelho, Proceedings 13th Annual Florida Ruminant Nutrition Symposium, pp 127-145).

According to the state of the art, stability issues are addressed by adding an additional amount of vitamin K3 in the feed in order to ensure the minimum K3 concentration. However, this results in additional costs.

The U.S. Pat. No. 5,128,151 discloses the use of physiologically tolerated organic or inorganic acids to improve the stability of MSB. One of the drawbacks of said approach is the use of a protective agent with no vitaminic activity. In fact, the addition of an exogenous compound with no vitaminic activity has been the main contributing factor to the continuous downward trend in the use of MPB as a source of K3 in animal feed. On the other hand, the upward trend in the use of MNB is mainly due to the fact that the exogenous inert compound in MPB (with no vitaminic activity) was replaced by niacinamide with B3 vitaminic activity. The comparison of the recommended amounts of vitamin B3 and vitamin K3 (expressed as MSB or MNB) shows that the weight ratio varies between 2 and 20 depending on the animal species (see FIG. 3).

As it may be seen, a formulation containing excess amounts of B3 as a protective agent presents the additional advantage of being the source of vitamin B3 that needs to be added to the premix in any case.

The joint addition of MNB or MSB along with nicotinamide (NA) and/or carbazochrome (CSS) to a mixture of herbicides and their inert support has been reported in the prior art (Japanese Patent Disclosure S58-206505). In this disclosure, the objective is to assure the coexistence of the herbicide along with the vitamin K3 and/or nicotinamide and/or carbazocrome after the scattering of the herbicide in order to diminish as much as possible the toxic effect of the herbicide on different aquatic species that will come into contact with the herbicide. Therefore, the objective of this prior art disclosure is neither to increase the stability of the vitamin K3 derivative in any solid mixture nor is the type of formulation prepared by the proposed combination of the ingredients adequate to form any protective barrier around the vitamin K3 derivative to increase its stability in a harsh environment such as feed premixes or during the severe conditions of operations such as pelletizing. In fact, the mixture of vitamin K3 derivative (MNB or MSB) and nicotinamide remains in a liquid form (dissolved in the herbicide) that has been impregnated on the substrate pellets. In this manner, the added nicotinamide does not exert any protective effect towards MNB or MSB in terms of stability.

### DESCRIPTION OF THE INVENTION

In contrast, the formulation according to the present invention is characterized inter alia by the fact that the excess NA confers a much higher stability to the vitamin K3 derivative especially in solid mixtures in which even stabilized forms of vitamin K3 such as MNB do not show the desired stability (see FIG. 2) by creating a physical barrier that protects the vitamin K3. This physical barrier is in the form of a continuous or non continuous layer that confers higher stability to the vitamin K3 derivative by decreasing its exposed surface area to the stability stress factors. The typical stress factors influencing the stability of vitamins in premixes, pelleting and storage are temperature, humidity, redox reactions and light.

The effects of some of these factors on the stability of some vitamin K3 derivatives as well as NA are presented in the Table 1 below (ref. <<Keeping Current (KC 9804), Vitamins in pet food>>, BASF Corporation, 1998).:

TABLE 1

| | Stress factor | | | | |
|---|---|---|---|---|---|
| | 1 | 2 | 3 | 4 | 5 |
| | Type of stress factor | | | | |
| | Vitamin premix | | | Petfood | Total |
| | without | Extrusion | Drying/ | storage | vitamin |
| | choline chloride | temperature | Enrobing | time | retention % |
| | Value of stress | | | | |
| | factor | | | | |
| Vitamin | 2 months | 105° C. | 180° C. | 8 months | 1 × 2 × 3 × 4 |
| MSB | 98% | 50% | 37% | 27% | 5% |
| MSBC | 98% | 60% | 52% | 30% | 9% |
| MPB | 100% | 65% | 70% | 42% | 19% |
| MNB | 100% | 70% | 76% | 47% | 25% |
| Niacinamide | 100% | 87% | 84% | 79% | 58% |

As it may be seen, each factor increases the degradation rate of the vitamin resulting in a lower stability. From the vitamin retention values it may also be noted that niacinamide results in a much higher stability compared even to MNB which is recognized as the most stable form of vitamin K3. Therefore, as mentioned before, the creation of the physical barrier of NA allows covering partly or entirely the exposed sensitive vitamin K3 derivative by a less sensitive layer of NA resulting in a higher stability of the formulation.

It should furthermore be noted that in the formulation according to the invention both components (vitamin K3 derivative and nicotinamide) are exclusively present in solid form.

The higher stability of products such as MPB (U.S. Pat. No. 3,281,69) and MNB (U.S. Pat. No. 4,577,019 and UK patent 2025976) has been related to the following factors:
Absence of crystallization water
Low water solubility
pH of saturated solutions are lower than 4.5

Contrary to these factors, the higher stability of the product according to the invention is based on the protective effect of the non-chemically bound excess NA layer in the final solid particles, based on the much higher chemical resistance of the NA molecule as indicated in Table 1 Apart from the stability issue, the vitamin K3 content in a premix is below 1 wt-%, which causes significant segregation or homogeneity problems in the vitamin premix. Both requirements, low segregation with relatively large particles (100-300 μm) in the range of the other compounds and high homogeneity with very small particles to guarantee a theoretically good distribution, can not be fulfilled at the same time.

Furthermore, small particles lead to lower stability due to a higher specific surface.

All the examples mentioned in the prior art show that there exists a need for other processes to enhance the stability of vitamin K3 derivatives and to improve the particle size distribution to overcome the drawbacks of the state of the art as well as a product obtainable by such a process. Said problems are surprisingly solved by the process according to the present invention as defined in the claims, which results in a product that is superior to the state of the art in the combination of:
1. higher stability;
2. better segregation properties; due to a formulation process which ensures a narrow particle size distribution resulting in particles with the same size compared to all other particles in the premix, segregation is contained.
3. better availability; even at low vitamin K3 derivative concentrations, a better spreading within the mixtures leads to a uniform distribution of the vitamin.
4. low dust; due to the formulation process, the new product has a low fraction of dust.
5. constant amount of substances in the formulation; due to a formulation consisting only of active substances, no additional filler or coating substance is needed.
Generally, said process is characterized by comprising the following steps:
   a) mixing of NA, the vitamin K3 derivative and water; and
   b) drying the mixture of step a);
   with the proviso that no organic or inorganic acid is used in step a) and b).

“Organic or inorganic acid” according to the present invention is defined as any Lewis acid or protic acid with a pKa<7.

The physical protective layer can be continuous or discontinuous, as long as a sufficient part of the surface of the vitamin K3 derivative particles is covered in order to achieve the technical advantages listed above.

This process differs substantially from the one reported in the UK patent 2025976 and U.S. Pat. No. 4,577,019 in that there is no chemical reaction between the vitamin K3 derivative used and the excess NA added.

It may be preferred to effect the removal of water in a spray dryer or spray-granulator.

Other preferred methods are high shear granulator or the combination of grinding, kneading, drying and breaking.

It may be preferred that the vitamin K3 derivative/NA mass ratio is between 2/1 and 1/100, particularly between 1/1 and 1/10.

“Derivative” according to the invention is used in its accepted chemical sense of describing a compound which arises from its parent compound by the replacement of one or more atoms with another atom or group of atoms.

It may be preferred that the vitamin K3 derivative is selected from the group consisting of MNB, MBP, MSBC and MSB.

Another object of the present invention is a NA-formulated vitamin K3 derivative, which is obtainable by a process according to the invention.

It may be preferred that the formulated vitamin K3 derivative particles have a size of at least 50 μm, preferably between 50 and 1000 μm and most preferably between 100 and 400 μm.

### BRIEF DESCRIPTION OF THE DRAWINGS

FIG. 1 is a graph which shows comparative accelerated stability test of formulated MNB according to the invention and pure MNB.

### EXAMPLES

The invention will be further described by the following, non-limiting examples.

### Example 1

### Spraygranulation

MNB was spray-granulated with a 40% (w/w) NA solution (heated to 60° C.) at a temperature at 60° C. in a laboratory granulator (Aeromatic). After the experiment, the ratio of MSB:NA was measured to 1:0.87 by HPLC. For further investigations, also the fraction between 100 μm and 315 μm was isolated.

### Example 2

### Combination of Grinding, Kneading, Drying and Breaking

MNB and NA (ratio 1:2) were grinded in a ball mill (Analysette Kugelmühle), mixed together with water in a kneader (2 h, ˜15% water, 25° C.). Afterwards, the product was dried at 50° C. and 15 mbar for 16 h in a vacuum drying oven. The product was grinded with a mortar and fractioned in a vibration sieve between 100 μm and 315 μm.

### Example 3

### Accelerated Stability Test

The matrix for the stability tests of NA-formulated MNB is shown in Table 2. The composition of the premix is shown below ‘premix’.

TABLE 2
Test matrix for vitamin K3 stability tests (“niacin” means
nicotinamide)

| substances | content |
|---|---|
| premix oligo-element | 15.2% |
| choline chloride 50% | 24.3% |
| copper sulfate | 14.2% |
| CaCO3 | 37.4% |
| premix | 8.9% |
| A | 6.46% |
| D3 | 3.09% |
| E 50% | 29.70% |
| B1 | 0.99% |
| B2 | 2.97% |
| Pantothenic acid | 7.42% |
| K3 | 0.00% |
| B6 | 0.99% |
| B12 0.1% | 9.90% |
| niacin | 0.00% |
| folio acid | 0.49% |
| C | 25.61% |
| (ashes) | 12.37% |

For the accelerated stability test, the sample of the mixture above was divided on day 0 in flasks for HPLC-preparation. These flasks were stored in a climate chamber at a constant atmosphere of 25° C. and 65% moisture. For each data point, two samples were taken.

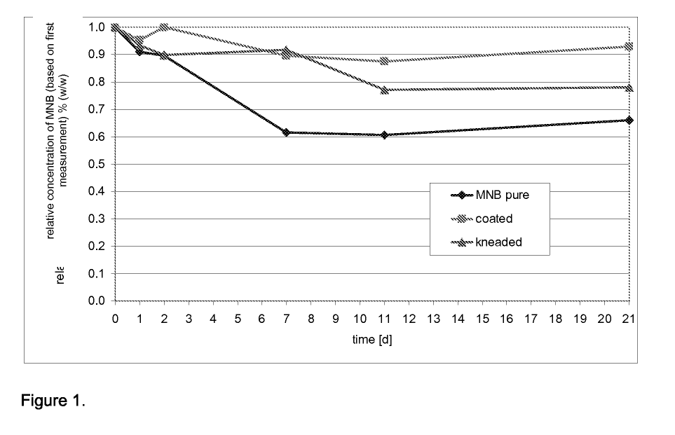

The products were compared in the accelerated stability test with the standard mixture of MNB with NA and the other substances as presented above. The results are shown in FIG. 1.

The experiments indicate a better stability of NA-pre-formulated MNB than pure MNB. The NA coating shows better values than just mixing and granulating. After 11 days, the MSB-concentration remains constant in the range of the accuracy of measurement.

1. Nicotinamide (NA) formulated vitamin K3 derivative particles, whereby the NA forms a physical protective layer on the vitamin K3 derivative particles.

2. Nicotinamide (NA) formulated vitamin K3 derivative particles according to claim 1, whereby said particles have a size of at least 50 μm, preferably between 100 and 400 μm and most preferably between 200 and 350 μm.

3. Nicotinamide (NA) formulated vitamin K3 derivative particles according to claim 1, whereby the vitamin K3 derivative is selected from the group consisting of MNB, MBP, MSBC and MSB.

4. Product according to claim 1, whereby the vitamin K3 derivative/NA mass ratio is between 2/1 and 1/100, particularly between 1/1 and 1/10.

5. Process for the production of NA formulated vitamin K3 derivative particles according to claim 1, comprising the following steps:
a) mixing of NA, the vitamin K3 derivative and water; and
b) drying the mixture of step a);
with the proviso that no organic or inorganic acid is used in step a) and b).

6. Process according to claim 5, whereby the removal of water after mixing is effected in a spray dryer or spray-granulator.

7. Process according to claim 5, whereby the mixing and formulation is conducted in a high shear granulator and drying is conducted in a convective or contact dryer.

8. Process according to claim 5, whereby the process is a combination of grinding, kneading, drying and breaking.

9. Process according to claim 5, whereby the vitamin K3 derivative/NA mass ratio is between 2/1 and 1/100, particularly between 1/1 and 1/10.
